(19) United States
(12) Patent Application Publication
(10) Pub. No.: US 2002/0018999 A1
(43) Pub. Date: Feb. 14, 2002

(54) Methods for characterizing polymorphisms

(76) Inventors: Jan Henriksson, Uppsala (SE); Jorgen Jonsson, Uppsala (SE); Torbjorn Norberg, Uppsala (SE)

Correspondence Address: GEMINI GENOMICS, INC., 189 WELLS AVENUE, NEWTON, MA, 02459, US

(21) Appl. No.: 09/792,415

(22) Filed: Feb. 23, 2001

(30) Foreign Application Priority Data
Feb. 24, 2000 (GB) 0004396.8
Oct. 4, 2000 (GB) 0024328.7

(51) Int. Cl. C12Q 1/68

(52) U.S. Cl. 435/6

(57) ABSTRACT

Methods are described for analyzing at least one polymorphic site in a biological sample containing at least one single-stranded template, including the steps of: combining the biological sample with a primer specific for each polymorphic site in each template and a primer extension preparation, to form an assay mixture; wherein the preparation includes: chain terminating nucleotides forming a first nucleotide class, chain elongating nucleotides forming a second nucleotide class such that the second nucleotide class does not include a nitrogenous base present in the first nucleotide class, and a template-dependent nucleic acid polymerase; incubating the mixture for a time and at a temperature sufficient to extend each primer by addition of at least one nucleotide; and determining the size of each extended primer. Kits suitable for practising the invention are also provided.

### RELATED APPLICATION(S)

This application claims priority to GB 0004396.8, which was filed on Feb. 24, 2000, and GB 0024328.7, which was filed on Oct. 4, 2000.

The entire teachings of the above application(s) are incorporated herein by reference.

### BACKGROUND OF THE INVENTION

Genetic variation, observed as polymorphisms, in the human genome is the subject of extensive research in the biomedical and pharmaceutical industries. Such variation is the source of each human's individuality, and, as such, can provide for forensic markers used in, for example, determining paternity and identity. However, when polymorphisms occur in certain genes, they can cause or contribute to diseases, or can impact an individual's response to therapeutic drugs.

The publication of the first draft of the human genome, and the international effort to map polymorphisms in the human genome, represent the first step toward refining design and clinical testing of new pharmaceuticals. A recent report sponsored by The SNP Consortium, Ltd. estimates that by 2005 at least 50% of all clinical trials will involve genotyping, for example, to assist in trial design and subject recruitment. The ability to predict drug response and/or possible side effects in an individual on the basis of genotyping will significantly impact the cost and accuracy of clinical trials.

The SNP Consortium report has estimated that in order to implement genome wide scans on large clinical populations, technology must be capable of generating at least one million genotypes per day. This number is based on screening clinical populations of up to 1000 patients for 100,000 discreet single nucleotide polymorphisms (SNPs) over a trial period of three months. Thus, there is a need for a reliable and economic method for genotyping. The SNP Consortium identifies the following as critical factors that must be resolved by 2005: low cost (3-5¢ per genotype), high throughput (10⁵ genotypes per day), sensitivity (≦1 ng DNA per genotype), scalability (high throughput for discovery, lower throughput for focused research and assay development), and iteration time between runs (hours to days).

A number of methods are known for assaying polynucleotides for the presence or absence of a particular nucleotide at a particular genetic locus. Such methods are disclosed, for example, in U.S. Pat. Nos. 4,656,127; 4,851,331; 5,679,524; 5,834,189; 5,849,542; 5,853,979; 5,869,242; 5,876,934; 5,908,755; 5,912,118; 5,928,906; 5,952,174; 5,976,802; 5,981,186; 6,004,744; 6,013,431; 6,017,702; 6,046,005; 6,087,095; and 6,117,634. However, currently available technology cannot meet the goals outlined above by The SNP Consortium, so a need exists for improved methods for detecting polymorphisms in biological samples.

Traditional methods for determining the sequence of DNA (“sequencing” methods) involve, predominantly, either a chain-terminating enzymatic reaction (Sanger et al., Proc. Natl. Acad. Sci. USA, 74:5463-5467 (1977)) or the Maxam-Gilbert method (Maxam, A. and Gilbert, W., Proc. Natl. Acad. Sci. USA, 74:560-564 (1977)). In the first method, labeled DNA fragments are synthesized enzymatically by reading a template that has been provided. Fragments are generated when a chain “terminating” nucleotide (typically a “dideoxynucleotide,” which lacks the 3′ hydroxyl group necessary to allow for further polynucleotide extension) is incorporated into the DNA strand being synthesized, thus terminating synthesis. With the advent of thermostable polymerases, e.g., polymerases isolated from thermophilic bacteria such as, for example, Thermus aquaticus, Thermotoga maritima, Thermotoga strain FjSS3-B.1, Thermosipho africanus, Thermus thermophilus, Thermus favus, Thermus ruber, Thermoplasma acidophilum, Sufolobus acidocaldarius, Bacillus caldotenax, Bacillus stearothermophilus, Methanobacterium thermoautotropicum, Thermococcus litoralis and Pyrococcus furiosus (as described in U.S. Pat. No. 6,077,664, the entire teachings of which are incorporated herein by reference), enzymatic sequencing involving thermocycling is also commonly used to determine nucleotide sequences.

Classical sequencing methods identify the precise order of nucleotides of a DNA molecule. However, these methods have several limitations that make them inappropriate for use in diagnostics. These methods read one template sequence at a time and are fairly labor intensive. For example, screening 10⁵ genotypes per day is not a realistic possibility. However, by taking advantage of the fact that screening for polymorphisms, or “genotyping,” only require sequencing a few nucleotides at a specific locus, various “mini-sequencing” methods have been developed. Even with mini-sequencing methods, genotyping polymorphic sites has remained impractical. For example, U.S. Pat. No. 6,013,431 allows for the convenient sequencing of a polymorphic site only if there are fewer than four possible polymorphic variants at a polymorphic site, and the method described can only sequence one polymorphic site per reaction.

### SUMMARY OF THE INVENTION

The present invention relates to methods and compositions for characterization of polymorphisms at known genomic loci. In particular, the invention relates to high throughput methods and kits for identification of polymorphisms in samples from individuals. The types of polymorphisms that can be detected include the following: a single nucleotide polymorphism, an insertion, a deletion, an inversion, a repeat, a microsatellite repeat, and a substitution.

In one embodiment, the invention is directed to a method of analyzing at least one polymorphic site of interest in a biological sample containing at least one single-stranded template, including the steps of: combining the biological sample with a primer specific for each polymorphic site in the template and a primer extension preparation, to form an assay mixture. If multiple polymorphic sites on the template are to be analyzed, then more than one primer specific to each polymorphic site is used. It is important to note that, by using the methods described herein, multiplex assays can be performed where any combination of multiple primers and templates can be analyzed in a single assay. The primer extension preparation includes chain terminating nucleotides forming a first nucleotide class; chain elongating nucleotides forming a second nucleotide class such that the second nucleotide class does not include a nitrogenous base present in the first nucleotide class; and a template-dependent nucleic acid polymerase. The mixture is incubated for a time and at a temperature sufficient to extend each primer by addition of at least one nucleotide. After the incubation step, the size (e.g., length) of each extended primer is determined.

In particular embodiments, the template can be immobilized on a solid phase or the template can be in a solution. In one embodiment, the second nucleotide class includes a single nitrogenous base, nucleotides comprising two nitrogenous bases, or nucleotides comprising three nitrogenous bases. In one embodiment, multiple polymorphic variants (e.g., between 1 and 6 polymorphic variants) are detected at the polymorphic site.

To aid in detection, the primer or the first nucleotide class can be labeled. The label can be one of the following: a radiolabel, a fluorescent label, a magnetic label, or an enzymatic label. The determining step can be any method of detection, possibly in combination with a method for separating nucleic acid molecules, suitable for determining the size (e.g., length) of the primer extension fragment. In one embodiment, the determining step can include, for example, chromatography or electrophoresis. This embodiment can include one or more (e.g., repeated consecutively) loadings of a solid matrix suitable for electrophoresis or chromatography.

In another embodiment, the invention is directed to a kit for analyzing at least one polymorphic site in a biological sample containing at least one single-stranded template. The kit includes one or more of the following: a sequencing primer specific for each polymorphic site of interest in each template; one or more components of a primer extension preparation that includes chain terminating nucleotides forming a first nucleotide class, chain elongating nucleotides forming a second nucleotide class, and a template dependent nucleic acid polymerase. In a particular embodiment, the second nucleotide class can include the following: nucleotides with a single nitrogenous base, nucleotides with two nitrogenous bases, or nucleotides with three nitrogenous bases. The kit may also include a solid phase means for binding the templates.

The kit may optionally include at least one primer with one or more of the following retention moieties: a polypeptide, an oligonucleotide, a polyamine, a polysaccharide, an aliphatic moiety comprising between one and fifteen carbon atoms, or an aromatic moiety. In one embodiment, the primer or the first nucleotide class has a label. The label can be one of the following: a radiolabel, a fluorescent label, a magnetic label, or an enzymatic label.

In another embodiment, the invention is directed to a method of analyzing at least one polymorphic site in a biological sample containing at least one single-stranded template. The method includes the steps of combining the biological sample with a sequencing primer specific for each polymorphic site of interest on each template and a primer extension preparation to form an assay mixture. The preparation includes chain elongating nucleotides lacking one nitrogenous base that is complementary to one polymorphic variant present in the template strand at the polymorphic site, and a template-dependent nucleic acid polymerase preferably with no nuclease activity, but having proofreading activity. The mixture is incubated for a time and at a temperature sufficient to extend the primer by addition of at least one nucleotide. Following the extension step, the size of the primer extension product is determined.

In other embodiments, the invention is directed to a kit for analyzing at least one polymorphic site in a biological sample containing at least one single-stranded template. The kit can include one or more of the following: a sequencing primer specific to each polymorphic site in each template; a primer extension preparation that includes sets of chain elongating nucleotides, each set lacking one nucleotide complementary to one polymorphic variant present at the polymorphic site; and a template dependent nucleic acid polymerase.

Thus, as a result of the invention described herein, methods are now available for reliable and economic genotyping. The methods of the present invention have several advantages compared to other SNP scoring methods. First, the method produces a very high quality typing result. All variants of a specific polymorphic site are scored in a single reaction using a single terminator or no terminator, and the results are derived from a single size separation. Moreover, several types of polymorphisms can be detected, e.g., SNPs, small insertions and deletions, as well as microsatellites and other smaller DNA repeats. Second, multiplex analysis is possible. Multiple polymorphic sites located on one or more PCR fragments can be analysed simultaneously if sequencing primers of different lengths are used for different polymorphic sites in the same mini-sequencing reaction. Third, the method has a high flexibility regarding the sequencing platform. Standard, commercially available sequencing equipment and reagents can be used. Finally, the methods described herein facilitate medium to high throughput SNP scoring using various multiplexing methods and technological platforms.

### BRIEF DESCRIPTION OF THE DRAWINGS

FIG. 1 is a schematic representation of the principle of a mini-sequencing method described herein. Horizontal arrows indicate sequencing primers.

FIG. 2 is a graph depicting the results of an experiment in which one SNP was investigated in three samples using Cy5-labeled ddCTP as a terminator. Size standards are Cy5-labeled primers 13 and 75 nucleotides long (first and last peak in each sample).

FIGS. 3A and 3B show the results of adapting the mini-sequencing method described herein for multiplexing.

FIG. 3A is a graph depicting the results of an experiment in which four samples and 5 SNPs were subjected to mini-sequencing in a multiplex experiment using Cy5-labeled ddCTP as a terminator. The upper line corresponds to the theoretical location of SNP variant peaks, the first two corresponds to SNP 1, the third and fourth peaks to SNP 2 and so on. The peaks at 13 and 75 bases are size standards.

FIG. 3B is a table depicting the selection of samples shown in FIG. 3A and their genotypes. The expected length of each sequencing product is also indicated.

### DETAILED DESCRIPTION OF THE INVENTION

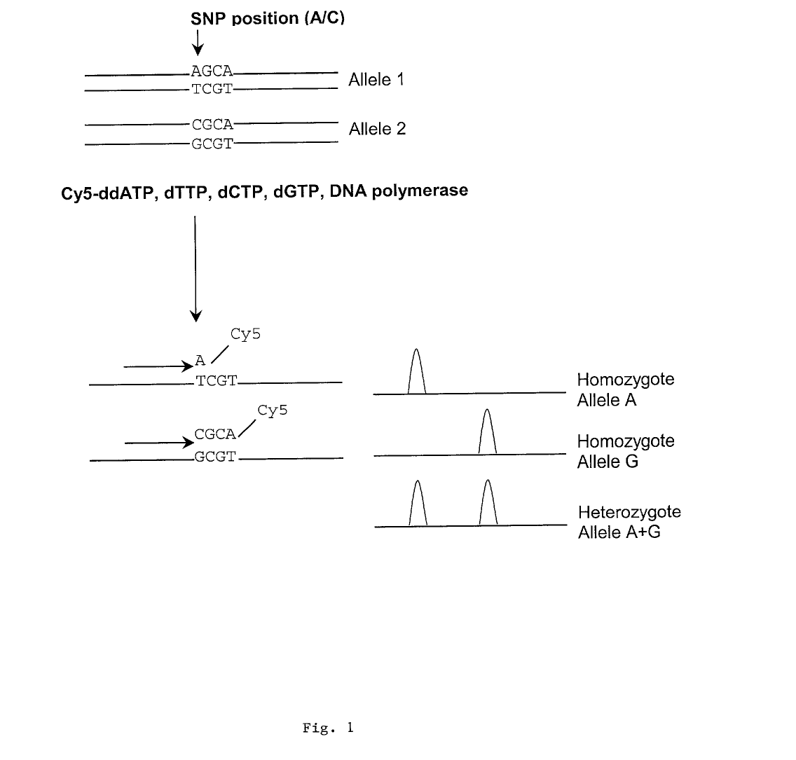

The present invention relates to methods and compositions for characterization of polymorphisms at known genomic loci. In particular, the invention relates to high throughput methods and kits for identification of polymorphisms in genomic DNA of individuals. The method of the present invention is a mini-sequencing/primer extension variant that uses a unique mixture of nucleotides (either labeled or un-labeled) to produce primer extension fragments of different length that are indicative of a particular polymorphic variant at a polymorphic site. Thus, a heterozygote sample will produce two extension products of different defined lengths (FIG. 1).

The present invention describes methods for producing primer extension fragments or products of various size depending on the particular nitrogenous base present at a polymorphic site. Conditions suitable for obtaining such products are satisfied in “primer extension preparations,” which can include, for example, any or all of the following: a primer specific for the template containing the polymorphic site, chain terminating nucleotides forming a first nucleotide class, chain elongating nucleotides forming a second nucleotide class, and a suitable polymerase. For example, if it is known that two different polymorphic variants can occur at a particular polymorphic site on a particular chromosome, then the method of the present invention can identify which nucleotide is present at that polymorphic site, thus identifying which polymorphic variant is present. A particular feature of the method described by the present invention is that a specific primer extension product for each polymorphic variant, i.e., one primer extension reaction identifies all polymorphic variants using only one terminator, as opposed to other methods, such as that described in U.S. Pat. No. 6,013,431, that only show the presence of a signal indicating the presence of a particular polymorphic variant, and infer the presence of a different polymorphic variant if no result is obtained. For example, if a heterozygous sample is analyzed (“heterozygote” as is used herein denotes the presence of different polymorphic variants at the same polymorphic site on an individual's matching chromosome pair), primer extension products of different sizes would be generated—one for each polymorphic variant. Thus, the methods of the present invention have an internal control whereby an investigator will be able to determine directly if a particular polymorphic variant is present or if the reaction failed.

As defined herein, a “template” is a nucleic acid. More specifically, the template can be of any size suitable for primer hybridization that allows for the analysis of polymorphic variants. For example, templates can be PCR fragments ranging in size from about 100 bases to about 5 kilobases, or restriction fragments ranging in size up to about 20 kilobases. Any nucleic acid may be analyzed using the methods and kits of the invention, so long as the nucleic acid, if double-stranded, is rendered single-stranded prior to analysis. Methods for separating strands of nucleic acids are well known to those of skill in the art, and may include, without limitation, exposing the template to a temperature sufficient to melt the strands, exposing the template to alkali conditions, exposing the template to chemical denaturants, and the like. The template analyzed in accordance with the invention may be genomic DNA, cDNA, mRNA, tRNA, coding sequences, non-coding sequences, sense strand strands, antisense strands, and the like.

The template can be isolated from a biological sample from any suitable source. Specifically encompassed by the present invention are mammalian or human samples obtained from biological sources containing cells, obtained using known techniques, from body tissue (e.g., skin, hair, internal organs), or body fluids (e.g., blood, plasma, urine, semen, sweat). Other sources of biological samples suitable for analysis by the methods of the present invention are microbiological samples, such as viruses, yeasts and bacteria; plasmids; isolated nucleic acids; and agricultural sources, such as recombinant plants. The biological sample is treated in such a manner, known to those of skill in the art, so as to render the template molecules contained in the biological sample available for binding, hybridizing and/or use as a template in a polymerization reaction.

Preferably, the template is an isolated and purified nucleic acid. An “isolated” nucleic acid molecule, as used herein, is one that is separated from nucleotides that normally flank the containing the polymorphic site in nature. With regard to genomic DNA, the term “isolated” refers to oligonucleotides containing polymorphic sites that are separated from the chromosome with which the genomic DNA is naturally associated. Alternatively, isolated nucleic acids can be amplification products of nucleic acids, e.g., products of the polymerase chain reaction (hereinafter, “PCR”). Moreover, the template analyzed in accordance with the invention can be in a crude cell lysate. Preferably, the template is substantially free of other cellular material, or culture medium when produced by recombinant techniques, or chemical precursors or other chemicals when chemically synthesized. In other circumstances, the material may be purified to essential homogeneity, for example as determined by PAGE or column chromatography such as HPLC.

“Primers” are oligonucleotides that hybridize in a base-specific manner to a complementary strand of nucleic acid. The present invention utilizes primers capable of being extended by enzymatically adding nucleotides to the 3′ end of the primer, thus creating “primer extension fragments.” Primers can be any length suitable for specific hybridization to the template. Thus, a primer can be any oligonucleotide such that it hybridizes to the template sequence and allows for extension by at least one nucleotide. Such optimizations are known to the skilled artisan. Suitable primers can range from about 12 nucleotides to about 150 nucleotides in length. For example, primers can be 12, 14, 15, 16, 18, 20, 22, 24, 25, 26, 28, 30, 35, 40, 45, 50, 60, 70, 80, 90, 100, 125 or 150 nucleotides in length. Preferably, primers used in the methods and kits of the invention are 15 to 25 nucleotides in length.

Hybridizations can be performed under stringent conditions, e.g., at a salt concentration of no more than IM and a temperature of at least 25° C. For example, conditions of 100 mM Tris, pH 6.0 and 10 mM MgCl₂ at a temperature of 65° C. or equivalent conditions are suitable for primer hybridization to template specific sequences. Equivalent conditions can include 5× SSPE (750 mM NaCl, 50 mM Na-Phosphate, 5 mM EDTA, pH 7.4) and a temperature of 25-30° C., or equivalent conditions, are suitable for hybridization to sequences specific to particular polymorphic variants. Equivalent conditions can be determined by varying one or more of the parameters given as an example, as known in the art, while maintaining a similar degree of identity or similarity between the target nucleotide sequence and the primer or probe used. Defining appropriate (e.g., high stringency, medium stringency, low stringency) hybridization and wash conditions is within the skill of the art (Ausubel et al., Current Protocols in Molecular Biology, John Wiley & Sons, Inc., New York; Sambrook et al., Molecular Cloning. A Laboratory Manual, Cold Spring Harbor Laboratory Press).

For known polymorphic sites, a primer can be designed such that it anneals to a template sequence immediately adjacent to a polymorphic site. The primer is designed such that it is complementary to a portion of the template starting “n” nucleotides away from the polymorphic site to be analyzed, such that “n” is the number of nucleotides between the nucleotide hybridized to the 3′ end of the primer and the polymorphic site on the template; “n” can be any number greater than or equal to zero. The only limitation on “n” is that there can not be a nucleotide present in the sequence encompassed by “n” that would necessitate the insertion of a terminating nucleotide into the primer extension fragment or otherwise direct chain termination. Thus a primer suitable for use in the present invention can be complementary to a region of the template either immediately adjacent to the polymorphic site or to a region several nucleotides upstream of the polymorphic site, so long as the template does not contain a nitrogenous base complementary to the first nucleotide class between the region where the primer hybridizes to the template and the polymorphic site. In one embodiment, each primer can have a retention moiety such as one of the following: a polypeptide, an oligonucleotide, a polyamine, a polysaccharide, an aliphatic moiety comprising between one and fifteen carbon atoms, and an aromatic moiety.

Typically, four nucleotides (hereinafter referred to as “NTPs” or, when referring to deoxynucleotides, “dNTPs”) are required for extension, adenosine triphosphate (hereinafter, when referring to the deoxynucleotide, “dATP” or, more generally, “dA”), cytosine triphosphate (hereinafter, when referring to the deoxynucleotide, “dCTP” or, more generally, “dC”), guanosine triphosphate (hereinafter, when referring to the deoxynucleotide, “dGTP” or, more generally, “dG”), and thymine triphosphate (hereinafter, when referring to the deoxynucleotide, “dTTP” or, more generally, “dT”). Thus, the method of the present invention allows for two classes of nucleotides: a first class that does not allow for chain extension (“chain terminators”). The chain terminating nucleotides of the invention do not comprise a 3′ hydroxyl group, which would be required for chain elongation by a DNA polymerase. The second class of nucleotides does allow for chain extension (“chain elongators”). The chain elongating nucleotides of the invention each comprise a 3′ hydroxyl group, which allows for chain elongation by a DNA polymerase. The chain elongating nucleotides of the invention that does allow for chain extension (“chain elongators”). The nucleotides present in the primer extension preparation each comprise a “nitrogenous base” such as, for example, adenine, guanine, hypoxanthine, cytosine, thymine, uracil, inosine, and the like.

In accordance with the present invention nucleotides of the first class can be optionally labeled. Labels can be radioactive, e.g., ³³P, ³²P, ³⁵S, ¹⁴C, ³H, ¹²⁵I; fluorescent, e.g., TAMRA (5[6]-carboxytetramethylrhodamine), ROX (5[6]-carboxy-X-rhodamine), JOE (6-carboxy-4,5′-dichloro-2′,7′-dimethoxyfluorescein), FAM (5[6]-carboxyfluorescein), R110, R6G, TET, HEX, NAN, ZOE, VIC, NED, PET, BigDye, fluorescein, rhodamine, Cy2, Cy3, Cy5, Cy5.5 and Texas Red (sulphorhodamine 101 acid chloride); enzymatic, e.g., horseradish peroxidase or alkaline phosphatase; or physical, e.g., labels that can be detected by interacting with other agents or labels that can be detected based on physical properties, e.g., electron spin states.

The invention relates in part to a method for producing a primer extension product whose length is dependent on the particular polymorphic variant present at a polymorphic site on a template molecule that encompasses the polymorphic site. For example, if dA or dC polymorphic variants (for the purposes of this example, allelic sequences refer to the sequence present in the strand that is being synthesized; thus, the template sequence contains the complementary dT or dG, respectively) are possible at a particular polymorphic site, the method of the present invention, for example, would provide a primer such that the primer hybridizes immediately adjacent to the polymorphic site, such that the 3′ end of the primer is adjacent to the polymorphic site. Thus, when the primer is extended, preferably by a suitable polymerase in an appropriate primer extension preparation, the first base that will be added to the 3′ end of the primer is the one complementary to the polymorphic site on the template. For this example, primer extension would be terminated if one polymorphic variant was present, or extension would continue if the other polymorphic variant is present.

The choice between extension or termination depends on the primer extension preparation. As described herein, the invention encompasses methods for choosing an appropriate primer extension preparation. For example, when analyzing a template that could contain either of the polymorphic variants dA or dC, such a preparation could include three dNTPs and one chain terminating nucleotide (for example, a dideoxynucleotide; “ddNTP”) corresponding to one of the possible allelic versions. In this example, ddATP, dCTP, dGTP, and dTTP are present. If the dA polymorphic variant is present, then synthesis terminates after the addition of ddATP. If the dC polymorphic variant is present, then synthesis continues past the polymorphic site until the next dA site is reached and a ddATP is added, thus terminating extension. Thus, fragments of two different and predictable lengths are generated. Alternatively, primer extension fragments can be obtained when one or more dNTPs are omitted from the reaction mix (see Example 3). For example, for the template described above, if the primer extension preparation contained only dCTP, dGTP and dTTP, then primer can be designed such that it hybridizes to the template “n” nucleotides away from the polymorphic site. As described above, the sequence encompassed by “n” does not include a nucleotide that would necessitate the insertion of a dATP, since dATP is not present in the primer extension preparation and would cause chain termination prior to reading the polymorphic site. In this example, primer extension would terminate after being extended “n” bases if the dA polymorphic variant is present at the polymorphic site, but would continue past “n” bases if the dC polymorphic variant is present. Thus, in both cases, different size primer extension fragments are generated—the size being dependent on the specific polymorphic variant present at a polymorphic site. Since determining the size of primer extension products leads to determination of genotype, the effect of intrinsic exonuclease activity of polymerases may affect the primer extension product. Preferably, a polymerase having proofreading activity is used when the primer extension reaction is performed by omitting the terminator.

Overall, by determining the size (i.e., length) of the primer extension fragments, the particular polymorphic variant present in a sample can be determined. Primer extension fragments are separated and either compared to each other, an internal size standard, or both. For example, any of a number of electrophoretic methods are available to one of skill in the art to separate primer extension products. These methods separate nucleic acids based essentially on the size of the nucleic acid, typically with larger nucleic acid molecules migrating more slowly through a solid phase medium than smaller nucleic acid molecules. By including nucleic acid molecules of a known length, the exact size of the primer extension products produced by a method of the present invention can be determined. In order to determine where in a solid phase a nucleic acid migrates, methods of detecting nucleic acids are also provided.

The template may be analyzed after immobilization on a solid support, or analyzed in solution, for example, using a “cycle sequencing”-like protocol. In this embodiment, the template, if amplified, is purified away from the PCR primers used, and rendered single-stranded using any of the methods set forth above. The sequencing primer is added, and a thermostable DNA polymerase is employed to extend the sequencing primer by at least one nucleotide. The template/primer duplex is denatured by exposure to elevated temperature, and after cooling, the template and primer are allowed to re-anneal, and the primer extension reaction is allowed to proceed. Additional rounds of denaturation, annealing, and extension are performed as desired. In this way, the method of the invention may be increased at the discretion of the user. Instruments appropriate for performing the “cycle sequencing” embodiment of the invention are commercially available. For example, the MegaBase MB1000™ or MB500, available from Amersham Pharmacia Biotech AB, Sweden, or the ABI Prism 9700™, ABI Prism 3100™, ABI 377™, or ABI 310™, all available from Applied Biosystems (Foster City, Calif.) may be employed to practice the cycle sequencing embodiment of the method of the invention.

The method of the present invention involves producing primer extension fragments of different lengths depending on which polymorphic variant is present at a polymorphic site. Fragments can be separated by size (e.g., length) by a number of methods commonly known in the art in order to determine which of the two allelic versions is present. For example, electrophoresis, chromatography, gel filtration, and HPLC are all methods suitable for use in the present invention to separate primer extension fragments. After separation, nucleic acids can be detected by any number of staining methods, e.g., such as treatment with ethidium bromide, or through sequence-specific hybridization methods known in the art. Alternatively, nucleic acids can have one or more modified chemical groups that serve as a detectable label. The invention also describes sequencing methods that include a primer or terminating nucleotide that has a detectable label. These molecules can be used to detect the presence of a fragment that has the labeled primer or terminating nucleotide incorporated into it.

The size of detected fragments is determined, as is known in the art, by a comparison of known size standards or by internally comparing fragments to each other. For example, if fragments are electrophoresed through a solid matrix such as, for example, a polyacrylamide gel, oligonucleotides of known length can be loaded onto the gel. Thus, a plot can be generated as size versus gel migration. In determining the migration of the primer extension fragments, the size can also be determined. Alternatively, if two different primer extension products of different length are expected, migration rates of fragments will indicate the size of a fragment relative to others contained in the sample. Since the size of primer extension fragments is knowable based since the template sequence is known, a plot can be generated for size versus migration rate. Alternatively, fragment size can be determined by physical means, such as, for example, mass spectrometry.

The present invention overcomes limitations of other sequencing and mini-sequencing methods in that it can be adapted as a “high-throughput” method. “High-throughput,” as used herein when referring to sequencing methods, denotes the ability to process and screen a large number of nucleic acid samples and a large number of target sequences within those samples in a rapid and economical manner. High-throughput mini-sequencing of polymorphisms can be achieved through “multiplexing”—the ability to sequence more than one polymorphism at a time. The present invention lends itself, for example, to at least four, without limitation, different types of multiplexing: the ability to analyze more than one polymorphic site in more than one DNA template using the same terminator (or no terminator); the ability to analyze more than one of polymorphic site on a DNA template using the same terminator (or no terminator), the ability to detect a polymorphism on more than one DNA template using the same terminator (or no terminator), and the ability to detect multiple possible polymorphic variants (i.e., a plurality of polymorphic variants—see Example 4) at a particular polymorphic site using the same terminator (or no terminator). The capacity for mutliplexing canbe increased by using several different fluorophores as fluorescent labels.

One method described by the present invention allows for the addition of primers of different sequence. In this way, more than one polymorphic site can be typed. For example, a primer 15 nucleotides in length can be used to detect possible polymorphic variants at a first polymorphic site, while a primer 25 nucleotides in length can be used to detect possible polymorphic variants at a second polymorphic site, while a third primer 35 nucleotides in length can be used to detect possible polymorphic variants at a third polymorphic site, and so on. Depending on the specific polymorphic variants present at the polymorphic sites, different sized primer extension products are generated, and, thus, used to type the sample at each polymorphic site (see Example 2).

The mini-sequencing methods described herein allow for multiplexing when the template is attached to a solid matrix. Methods for attaching a nucleic acid template to a solid matrix are well known in the art, as are suitable solid matrices. If the template is immobilized, then particular primers can be used to mini-sequence a specific set of polymorphic sites, and, afterwards, the template, still attached to the solid matrix, can be washed and prepared for another round of mini-sequencing with the same or a different set of primers, thus allowing for typing a different set of polymorphic sites.

Another way the method described by the present invention can be used in a multiplexing assay is by detecting several possible polymorphic variants at a single polymorphic site. In some cases, there are only two possible polymorphic variants at any given locus. However, an advantage of the present invention is that it can detect several different polymorphic variants at a given locus (see Example 4). For example, one of several types of polymorphisms could be possible at a particular polymorphic site (e.g., any of the four SNP's, deletion polymorphisms, insertion polymorphisms). In addition to detecting specific polymorphic variants based on primer extension fragment size, methods described herein can detect multiple specific polymorphic variants at a polymorphic site by using differentially labeled terminating nucleotides, specific labels indicating particular polymorphic variants.

Yet another feature of the method described by the present invention is the ability to detect a range of polymorphisms in a sample containing multiple templates. The result of such an analysis, for example, provides a description of the range of polymorphisms possible at a particular locus within a population.

In another embodiment, the present invention relates to a kit for detecting, using the mini-sequencing methods described herein, the genotype of a sample. The kit comprises at least one container having disposed therein the above-described reagents necessary for forming primer extension fragments of various size depending on the particular polymorphic variant present in the sample. In a preferred embodiment, the kit includes other containers comprising wash reagents and/or reagents capable of detecting primer extension fragments generated as a result of the primer extension reaction. Examples of detection reagents include, but are not limited to radiolabels, enzymatic labels (horseradish peroxidase, alkaline phosphatase), affinity labeled labels (biotin, avidin, or streptavidin), fluorescent labels, and the like.

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container that will accept the test sample, a container that contains the primers used in the assay, containers that contain wash reagents (e.g., phosphate buffered saline, Tris buffers, and the like), and containers that contain the reagents used to detect the primer extension fragments. Instructions for use of the kit will also be included.

One skilled in the art will recognize that the reagents allowing for the practice of the methods described in the present invention can readily be incorporated into one of the established kit formats that are well known in the art.

The following Examples are offered for the purpose of illustrating the present invention and are not to be construed to limit the scope of this invention. The teachings of all references cited herein are hereby incorporated herein by reference.

### EXAMPLE 1

Mini-sequencing Method for the Detection of Polymorphisms.

The polymorphic positions used in these Examples are as shown in Table 1 below.

TABLE 1

| | | GenBank | Poly- | | |
|---|---|---|---|---|---|
| | | accession | morphic | | PCR |
| Gene | Symbol | No. | position | Variation | fragment |
| Angiotensin II | AGTR1 | U07144 | ATR 2415 | R = A/G | ATP f12R |
| receptor type 1 | | | | | |
| Angioten- | AGT | X15323 | AGR:1218 | R | ANP f8R |
| sinogen | | | | | |
| Beta I | ADRB1 | X69168 | B1P.2577 | Y = C/T | B1P f4F |
| adrenergic | | | | | |
| receptor | | | | | |
| Beta 1 | ADRB1 | J03019 | B1R:231 | R | B1R f7R |
| adrenergic | | | | | |
| receptor | | | | | |
| Beta 1 | ADRB1 | J03019 | B1R:1251 | S − C/G | B1R f6F |
| adrenergic | | | | | |
| receptor | | | | | |
| Beta 2 | ADRB2 | M15169, | B2P^{.}1120 | S | B2P f2F |
| adrenergic | | J02728, | | | |
| receptor | | M16106 | | | |
| Beta 2 | ADRB2 | Y00106 | B2R'1316 | M = A/C | B2R f3F |
| adrenergic | | | | | |
| receptor | | | | | |
| Beta 2 | ADRB2 | Y00106 | B2R:2068 | Insertion/ | B2Rf6F |
| adrenergic | | | | deletion | |
| receptor | | | | | |

Several commercially available sequencing kits providing reagents and apparatus are available. Solid phase sequencing with AutoLoad™ (Amersham Pharmacia Biotech AB, Sweden) combs and different dNTP/ddNTP mixtures were used to analyze several different SNPs. Sequenced products were separated and identified using the ALFexpress™ (Amersham Pharmacia Biotech AB, Sweden) instrument. To increase throughput, short glass plates and gels were used instead of glass plates of regular size.

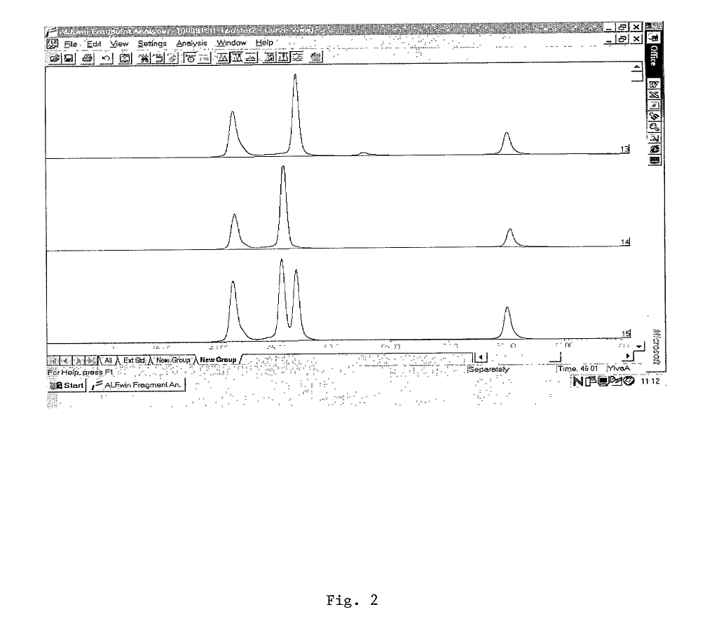

Initial experiments using the dye terminators Cy5-ddCTP and T7 DNA polymerase confirmed that the mini-sequencing method worked not only with dye primers but also with dye terminators. For the optimization experiments of defining nucleotide concentrations the dye terminator Cy5-ddCTP was used. Testing a dilution series of a nucleotide mix (equal amounts of ddNTP and dNTPs) showed that dilution down to 1:600 (i.e., 1.7 mM per nucleotide or 7 pmol per nucleotide and reaction) was giving reliable results and acceptable signal levels (≧10%). Three different polymerases, T7 DNA polymerase (T7), Thermo Sequenase I (TSI) and Thermo Sequenase II (TSII), were tested using Cy5-labeled ddCTP. All enzymes were used at a concentration of 6 u/reaction. All initial experiments were performed using optimal conditions for T7, i.e., 42° C. (heat block temperature), pH 7.6 and DMSO, which are not optimal conditions for TSI and TSII. In later experiments with TSI and TSII 65° C. (heat block temperature) and other buffers were used. All three enzymes were suitable, although TSI and TSII generated results of higher quality. In the T7 experiments the curves were less distinct and tailing was present in many cases. Thus, all following experiments with dye terminators have been performed with either TSI or TSII. The conditions were successfully confirmed with the three other bases as terminators. For each terminator one to five different PCR-fragments (one to five polymorphisms) were investigated in eight different samples (homozygote and heterozygote samples were present for each polymorphism). FIG. 2 shows an example of three polymorphic samples detected by the mini-sequencing method using TSI.

The initial experiments were made on long ALFexpress™ gels (Amersham Pharmacia Biotech AB, Sweden), although any suitable gel can be used. Short ALFexpress™ gels are preferred because of their shorter running times and the short elongation products of the mini-sequencing method described herein. The aims of the experiments were to evaluate the resolution of the peaks on a short gel in relation to the resolution on a long gel, and to develop an optimal throughput on a short gel. The results showed that short gels could be used instead of long gels without any decrease in resolution. In all further experiments described herein, short gels have been used. Routinely, short ALFexpress™ gels were used with three consecutive loadings.

Up to seven different fragments have been tested in multiplexing experiments. Of the seven polymorphisms, six were interpretable whereas the seventh, due to unexpected migration speed migrated together with a size standard. Sequencing primer lengths used were between 13 and 60 nucleotides. Multiplexing of, for example, four to six polymorphisms is now done routinely.

For the polymorphism mini-sequencing assay, only one reaction was used for each template-containing sample instead of the four reactions typically used during standard enzymatic sequencing (Sanger, F. et al., Proc. Natl. Acad. Sci. USA. 74:5463-7), and the nucleotide mixture was changed in the way that one of the four nucleotides is entirely replaced with chain terminating ddNTPs. The concentration of each nucleotide, ddNTPs and dNTPs alike, in the standard polymorphism typing assay experimental set up was 1 mM for each nucleotide. The solid phase method consists of four steps: binding, denaturation, annealing and extension.

When solid phase sequencing with AutoLoad combs was used, PCR products were bound to the combs using biotin and streptavidin and denatured under alkaline conditions. The sequencing step requires, for example added dNTPs , ddNTPs, a sequence specific primer capable of annealing adjacent to the polymorphic site, and a suitable polymerase. Under appropriate buffer conditions, the primer was extended and, depending on the specific sequence and the ddNTPs added, different length primer extension fragments were generated.

Specifically, when Cy5-labeled primers were used, all the reagents for annealing and extension reactions, except the enzyme, i.e., annealing buffer, extension buffer, nucleotides, enzyme-dilution buffer and Cy5-labeled primer, were removed from storage and left to thaw at room temperature. The tubes were vortexed and spun down before use. The Cy5-labeled primer was sensitive to light and is kept at a stock concentration of 100 mM at −20° C. Plastic dishes were prepared for use in the washing and denaturation procedure, three for TE (10 mM Tris, pH 7.5; 1 mM EDTA, pH 8.0), two for NaOH and two for Milli-Q water (Millipore, Bedford, Mass.), with tissues (low lint) between the vessels for blotting off excess liquid between the wash steps. The dishes were filled with each solution, to a depth of approximately 0.5 cm of 1× TE-buffer, freshly made 0.15 M NaOH and fresh Milli-Q water.

One tube was labeled for the annealing mix and one for the sequencing mix. The tubes were placed in a box filled with ice, to pre-chill. For a non-multiplexed assay using Cy5-labeled primers, the annealing mix and sequencing mix were prepared in Table 2 as follows:

TABLE 2

| Annealing mix: | 1x | | 22x | | 44x | | 88x | |
|---|---|---|---|---|---|---|---|---|
| H₂O | 17.3 | μL | 380.6 | μL | 761.2 | μL | 1522.4 | μL |
| Annealing buffer | 2 | μL | 44 | μL | 88 | μL | 176 | μL |
| Cy5-labeled | | | | | | | | |
| sequencing primer | 0.67 | μL | 14,67 | μL | 29.33 | μL | 58.67 | μL |
| (1 μM) | | | | | | | | |
| Total volume | ˜20 | μL | ˜440 | μL | ˜880 | μL | ˜1760 | μL |
| Sequencing mix: | 1x | | 22x | | 44x | | 88x | |
| H₂O | 10 | μL | 220 | μL | 440 | μL | 880 | μL |
| Annealing buffer | 2 | μL | 44 | μL | 88 | μL | 176 | μL |
| Extension buffer | 1 | μL | 22 | μL | 44 | μL | 88 | μL |
| DMSO | 2 | μL | 44 | μL | 88 | μL | 176 | μL |
| Enzyme dilution buffer | 0.25 | μL | 5.5 | μL | 11 | μL | 22 | μL |
| d/ddNTP mix* | 4 | μL | 88 | μL | 176 | μL | 352 | μL |
| T7 polymerase | 0.75 | μL | 16.5 | μL | 33 | μL | 66 | μL |
| (8 U/μl) | | | | | | | | |
| Total volume | 20 | μl | 440 | μl | 880 | μL | 1760 | μL |

* 2.5 mM each dNTP, 2.5 mM each ddNTP, 125 mM NaCl, 10 mM Tris pH 7.6. Nucleotide concentrations in the range of 1.7 mM to 100 mM have been used with successful results.

The tubes were put on ice before adding the enzyme. The enzyme was added to the sequencing mix, which was mixed using a vortex or pipette. After mixing, the mix was sedimented down to the bottom of the tube by centrifugation.

The combs were washed twice in TE by agitation and then left to stand for 30 seconds. Excess fluid from the combs was blotted by putting the combs on to low lint tissue. The combs were then denatured in fresh 0.15 M NaOH for five minutes. During these five minutes, it was convenient to add 20 μl annealing mix/well to a 40-well plate and then preincubate the plate for 1.5 minutes at 65° C. The combs were washed once in TE for 30 seconds and once in H₂O for 30 seconds, and excess fluid was blotted from the combs by putting the combs on a low lint tissue.

The combs were added to the pre-warmed annealing mix and incubated at 65° C. for 5 minutes. During the 5 minute annealing reaction, a 20 μL aliquot of the sequence mix was dispensed to each well of a new 40-well plate on ice. After the annealing incubation at 65° C. for 5 minutes, the plate was removed from the heater and cooled to room temperature for at least 1 minute and at most 5 minutes. The plate containing the sequencing mix was pre-incubated at 42° C. for 90 seconds. The cooled combs were washed with water, and excess fluid was blotted from the combs by putting them on low lint tissue. The sequencing reaction was initiated by placing the combs in the pre-warmed mix and incubating them at 42° C. for 5 minutes. The reaction was stopped by immersing the combs in TE buffer. The combs were stored in TE buffer at 4° C. until analysis.

### EXAMPLE 2

Multiplex Assay.

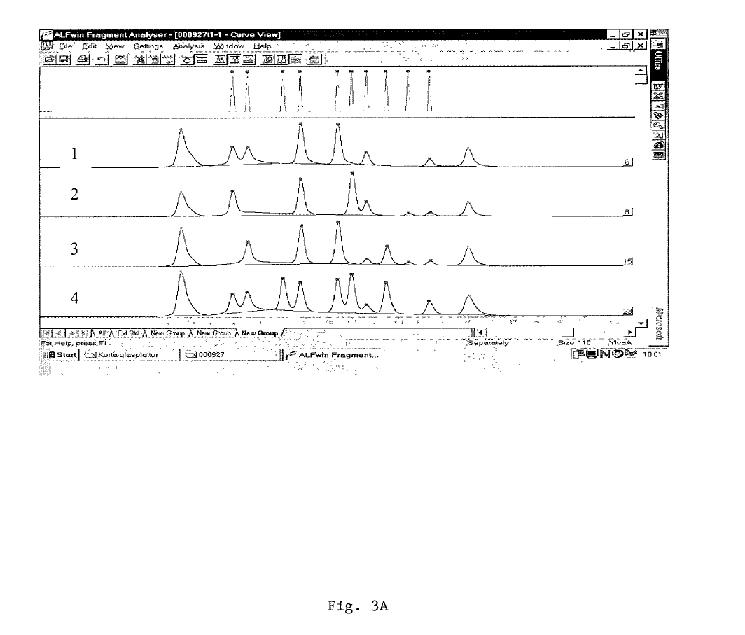

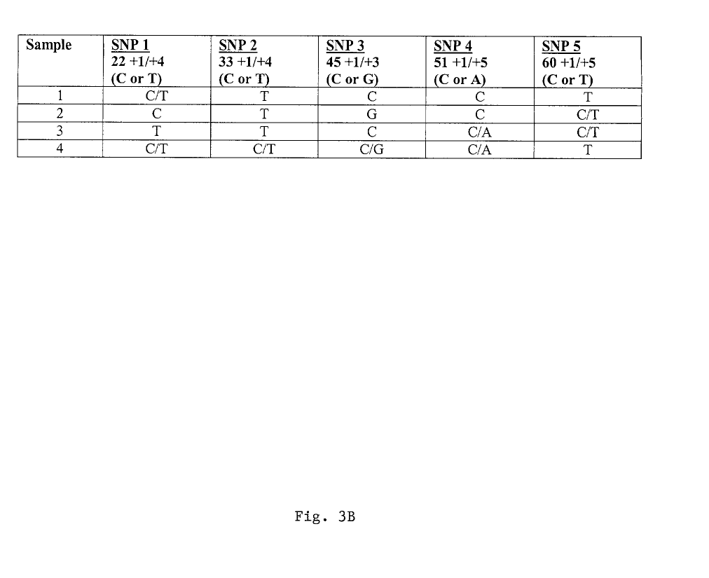

A multiplex analysis (FIG. 3A) of 5 SNPs (FIG. 3B) from three different genes was carried out in an 80 sample pilot with both Cy5-labeled primers (T7 DNA polymerase) and Cy5-labeled ddCTP (Thermo Sequenase I). The experiment included 78 samples and 2 negative controls. The samples had previously been fully sequenced on ALFexpress (FIG. 3A). Note that optimal conditions were used for both enzymes in these experiments (i.e., 42° C. (heat block temperature), pH 7.6, DMSO for T7 DNA polymerase, and 65° C. (heat block temperature), pH 9.5, no DMSO for TSI). The results are presented in Table 3. The success rate in the study was close to 100%.

TABLE 3

| | Correct | Incorrect | | |
|---|---|---|---|---|
| Cy5-label | genotypes | genotypes | Method failures | Success rate |
| Terminator | 387/390 | 2/390 | 1/390 | 99% |
| Primer | 381/390 | 9/390 | 0/390 | 98% |

Table 3. Success rate (compared to previous full sequencing results) from a pilot scale genotyping experiment including 78 samples and 5 SNP positions (i.e., 390 positions have been determined).

### EXAMPLE 3

Mini-sequencing With a Missing Nucleotide.

The mini-sequencing method described herein can be carried out by omitting one dNTP instead of adding one ddNTP. For example, in detecting an A/C SNP by omitting dATP nucleotides, no extension will not occur if an A is present in the SNP. If a C is present, extension will continue until the next A in the sequence template (where the reaction will stop due absence of dATP in the nucleotide mix). Thus, a heterozygous sample will produce two extension products of different, defined lengths.

### EXAMPLE 4

Mini-sequencing of a Polymorphic Site With More than Two Polymorphic Variants.

In the initial 5-multiplex design, there was a position (B2R:2068) in the beta 2 adrenergic receptor consisting of an insertion/deletion polymorphism (see Table 1). Re-evaluation of the full sequencing of this position revealed that it is more complex than previously anticipated. Instead of being a simple insertion/deletion, the position is a highly polymorphic site with at least six possible genotypes. The phenotypes are listed below in Table 4.

TABLE 4

| Polymorphic | |
|---|---|
| variant | DNA Sequence |
| 1 | . . . CTTTTAAAGACCCCCCCC---CCCAACAGAA |
| 2 | . . . CTTTTAAAGACCCCCCCCC--CCCAACAGAA |
| 3 | . . . CTTTTAAAGACCCCCCCCCC-CCCAACAGAA |
| 4 | . . . CTTTTAAAGACCCCCCCC--GCCCAACAGAA |
| 5 | . . . CTTTTAAAGACCCCCCCCC-GCCCAACAGAA |
| 6 | . . . CTTTTAAAGACCCCCCCCCCGCCCAACAGAA |

Using a primer with seven-3′ Cs (CTTTTAAAGACCCCCCC) and Cy5 labeled ddGTP, five of six polymorphic variants were detected in ten samples. The six polymorphic variants are detected by: +9 nt extension of polymorphic variant 1,+10 nt extension of polymorphic variant 2,+11 nt extension of polymorphic variant 3,+2 nt extension of polymorphic variant 4,+3 nt extension of polymorphic variant 5, and +4 nt extension of polymorphic variant 6.

### EXAMPLE 5

Sequencing Kits and Protocols

The following example describes protocols for analyzing polymorphic sites. The method is also referred to herein as the One Base Sequencing (OBS) method.

A “research kit” is described that enables one to use one base sequencing with dye terminators. The research kit consists of two parts—a “disposables kit” and a “reagents kit”.

The disposables kit for performing 400 OBS reactions includes 50×8-tooth streptavidin-coated sequencing combs (400 teeth total) and 40×40-well plates for sequencing reactions (1600 wells total).

The reagents kit for performing 400 OBS reactions is as follows (a separate kit should be available for each dye terminator, in total four different kits): 400× OBS kit (A), 400× OBS kit (C), 400× OBS kit (G), and 400× OBS kit (T). The concentrations of reagents listed in the Table 5 below are suggestions. However, the total reaction volumes for the annealing mix and sequencing mix should not exceed 20 μl, respectively.

TABLE 5

| | Concen- | | Per OBS | | 400xOBS | | 400xOBS | | 400xOBS | | 400xOBS | |
|---|---|---|---|---|---|---|---|---|---|---|---|---|
| Reagent | tration | | reaction | | kit (A) | | kit (C) | | kit (G) | | kit (I) | |
| Reagents for | | | | | | | | | | | | |
| annealing: | | | | | | | | | | | | |
| Annealing buffer | 10X | | 2 | μL | >800 | μL | >800 | μL | >800 | μL | >800 | μL |
| (AutoLoad ™ kit) | | | | | | | | | | | | |
| Reagents for | | | | | | | | | | | | |
| sequencing: | | | | | | | | | | | | |
| ThermoSequenase I | 12 | u/μL | 0.5 | μL (6u) | >200 | μL | >200 | μL | >200 | μL | >200 | μL |

| | | | | | | | | | | | |
|---|---|---|---|---|---|---|---|---|---|---|---|
| Reaction buffer | 10X | 2 | μl | >800 | μL | >800 | μL | >800 | μL | >800 | μL |
| (ThermoSequenase | | | | | | | | | | | |
| Cy5 terminator kit) | | | | | | | | | | | |

| | | | | | | |
|---|---|---|---|---|---|---|
| Cy5 ddA OBS mix | 2.5 | μM each | 4 | μL | >1600 | μL |

| Cy5 ddATP, dCTP, | nucleotide | | (10 | pmol/nt) | | |
|---|---|---|---|---|---|---|
| deazaGTP, dTTP | | | | | | |
| Cy5 ddC OBS mix: | 2.5 | μM each | 4 | μL | >1600 | μL |

| | | | |
|---|---|---|---|
| Cy5 ddCTP, dATP, | nucleotide | (10 | pmol/nt) |
| deazaGTP, dTTP | | | |

| | | | | |
|---|---|---|---|---|
| Cy5 ddG OBS mix: | 2.5 | μM each | >1600 | μL |

| CyS ddGTP, | nucleotide | | (10 | pmol/nt) | | |
|---|---|---|---|---|---|---|
| dATP, dCTP, dTTP | | | | | | |
| Cy5 ddT OBS mix: | 2.5 | μM each | 4 | μL | >1600 | μL |

| | | | |
|---|---|---|---|
| Cy5 ddTTP, dATP, | nucleotide | (10 | pmol/nt) |
| dCTP, deazaGTP | | | |

Buffers:

| | |
|---|---|
| 1 × BW buffer | 1M NaCl, 5 mM Tris-HCl pH 7.5, 0.5 mM EDTA |
| 1 × TB | 10 mM Tris-HCl pH 8, 1 mM EDTA |

The heat blocks are set to 65° C. The number of combs that are required (eight samples per comb) are marked in a convenient way. Opened packages with combs are thoroughly sealed and stored at 4° C. Add two parts of 0.5× BW buffer to samples in the PCR plate, e.g., 80 μL 0.5× BW buffer to 40 μL PCR-product. Mix by pipetting carefully to avoid bubbles. For multiplex analysis, products from two or more different PCRs can be pooled. Put the PCR product(s) and BW buffer in to a 40-well plate (see Table 6). If the signal levels are too high or too low, the volume of that specific PCR product can be adjusted.

TABLE 6

| | | | PCR product diluted in BW- | |
|---|---|---|---|---|
| Degree of multiplexing | 0.5xBW-buffer | | buffer (see above) | |
| Singleplex analysis | 16 | μL | 1 × 4 | μL |
| 2 plex analysis | 12 | μL | 2 × 4 | μL |
| 3 plex analysis | 8 | μL | 3 × 4 | μL |
| 4 plex analysis | 4 | μL | 4 × 4 | μL |
| 5 plex analysis | 0 | μL | 5 × 4 | μL |

The combs are placed into the wells and left at 65° C. for 30 minutes. Take out some plastic dishes to use in the washing procedure, three for TE buffer, two for NaOH and two for Milli-Q water. The dishes are filled to approximately 0.5 cm depth with the appropriate solution, i.e., TE buffer, freshly made 0.15 M NaOH and fresh Milli-Q water. Label one tube for the annealing mix and one for the sequencing mix. Place the tubes in a box filled with ice, to pre-chill.

Prepare the annealing mix as described in Table 7 below (Note the differences between single and multiplex experiments).

TABLE 7
Single analysis

| Anneal- | | | | | | |
|---|---|---|---|---|---|---|
| ing | | | | | | |
| mix: | 1x | | 22x | | 44x | |
| H₂O | 17.33 | μL | 381.26 | μL | 762.52 | μL |
| Anneal- | 2 | μL | 44 | μL | 88 | μL |
| ing | | | | | | |
| buffer | | | | | | |
| Sequen- | 0.67 | μL | 14.74 | μL | 29.48 | μL |
| cing | | | | | | |
| primer | | | | | | |
| (1 mM) | | | | | | |
| Total | 20 | μL | 440 | μL | 880 | μL |
| volume | | | | | | |

Multiplex analysis

| Anneal- | | | | | | |
|---|---|---|---|---|---|---|
| ing | | | | | | |
| mix: | 1 X | | 22 X | | 44 X | |
| H₂O | 18 | μL | 396 | μL | 792 | μL |
| | (N × 0.67 | μL) | (N × 14.74 | μL) | (N × 29.48 | μL) |
| Anneal- | 2 | μL | 44 | μL | 88 | μL |
| ing | | | | | | |
| buffer | | | | | | |
| Sequen- | N × 0.67 | μL | N × 14.74 | μL | N × 29.48 | μL |
| cing | | | | | | |
| primers | | | | | | |
| (1 mM) | | | | | | |
| (N = | | | | | | |
| No. of | | | | | | |
| diff- | | | | | | |
| erent | | | | | | |
| assays | | | | | | |
| in | | | | | | |
| multi- | | | | | | |
| plexing) | | | | | | |
| Total | 20 | μL | 440 | μL | 880 | μL |
| volume | | | | | | |

Prepare the sequencing mix as described in Table 8 below.

TABLE 8

| Sequencing mix | 1x | | 22x | | 44x | |
|---|---|---|---|---|---|---|
| H₂O | 13.5 | μL | 297 | μL | 594 | μL |
| Reaction buffer (10X) | 2 | μL | 44 | μL | 88 | μL |
| Nucleotide mix (2,5 μM per | 4 | μl | 88 | μl | 176 | μL |
| nucleotide) | | | | | | |
| Thermosequenase I 12 u per μl) | 0.5 | μL | 11 | μL | 22 | μL |
| Total volume | 20 | μL | 440 | μL | 880 | μL |

Put the tubes on ice before adding the enzyme. The enzyme should be kept on a cold-block. After the 30 minutes incubation of PCR products in BW buffer at 65° C., the combs are washed twice in TE buffer by moving the combs around in the dish and then letting them stand for 30 seconds. Excess fluid from the combs is removed by putting the combs on to low lint tissue (Note: it is important that the combs not dry completely).

The combs are denatured in fresh 0.15M NaOH for 5 minutes. During the incubation, 20 μL annealing mix per well is added to a 40-well plate. The annealing mix plate is pre-incubated for 90 seconds at 65° C. The denaturation step is completed by dipping the combs in fresh 0.15M NaOH. The combs are then washed once in TE buffer for 30 seconds and once in H₂O for 30 seconds (Note: remove excess fluid from the combs by putting the combs on a low lint tissue for a second between steps).

The combs are then added to the pre-warmed annealing mix and incubated at 65° C. for 5 minutes. The enzyme is added to the sequencing mix and vortexes. It is important to avoid air bubbles in the mixture.

Dispense 20 μl of sequence mix per well to a 40-well plate. After incubating at 65° C. for 5 minutes, the plate is removed from the heater and cooled to room temperature for between 1 and 5 minutes.

The plate containing sequence mix is pre-incubated for 90 seconds at 65° C. The combs are washed in a dish containing Milli-Q H₂O and the liquid excess is removed by use of low lint tissue. The combs are placed in the pre-warmed mix and incubated for 5 minutes at 65° C. The plate with the combs is removed from the heater and placed in a 40-well plate with TE buffer. The combs are stored at 4° C. for 1-3 days.

Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of analyzing at least one polymorphic site in a biological sample comprising at least one single-stranded template, said method comprising the steps of:
a) combining the biological sample with a primer specific for each polymorphic site in each template and a primer extension preparation, to form an assay mixture; wherein the preparation comprises:
i) chain terminating nucleotides forming a first nucleotide class;
ii) chain elongating nucleotides forming a second nucleotide class, wherein the second nucleotide class does not comprise any nitrogenous base present in the first nucleotide class; and
iii) a template-dependent nucleic acid polymerase;
b) incubating the mixture for a time and at a temperature sufficient to extend each primer by addition of at least one nucleotide; and
c) determining the size of each extended primer.

2. The method of claim 1, wherein the template is immobilized on a solid phase.

3. The method of claim 1, wherein the template is in a solution.

4. The method of claim 1, wherein the second nucleotide class is selected from the group consisting of nucleotides comprising a single nitrogenous base, nucleotides comprising two nitrogenous bases, and nucleotides comprising three nitrogenous bases.

5. The method of claim 1, wherein multiple polymorphic variants are detected at the polymorphic site.

6. The method of claim 1, wherein the primer or the first nucleotide class is labeled.

7. The method of claim 6, wherein the label is selected from the group consisting of a radiolabel, a fluorescent label, a magnetic label, and an enzymatic label.

8. The method of claim 1, wherein the determining step is selected from the group consisting of chromatography and electrophoresis.

9. The method of claim 8, wherein the determining step is by electrophoresis, and comprises one or more loadings of a solid matrix suitable for electrophoresis.

10. The method of claim 1, wherein the primer is complementary to a region of the template immediately adjacent to the polymorphic site.

11. The method of claim 1, wherein the primer is complementary to a region of the template within about 100 bases upstream of the polymorphic site.

12. The method of claim 1, wherein each primer further comprises a retention moiety selected from the group consisting of a polypeptide, an oligonucleotide, a polyamine, a polysaccharide, an aliphatic moiety comprising between one and fifteen carbon atoms, and an aromatic moiety.

13. The method of claim 1, wherein each polymorphic site is selected from the group consisting of a single nucleotide polymorphism, an insertion, a deletion, an inversion, a repeat, a microsatellite repeat, and a substitution.

14. A kit for analyzing at least one polymorphic site in a biological sample comprising at least one single-stranded template, said kit comprising:
a) a sequencing primer specific for each polymorphic site in each template;
b) a primer extension preparation comprising:
i) chain terminating nucleotides forming a first nucleotide class;
ii) chain elongating nucleotides forming a second nucleotide class;
iii) a template dependent nucleic acid polymerase.

15. The kit of claim 11, wherein the primer or the first nucleotide class comprises a label.

16. The kit of claim 15, wherein the label is selected from the group consisting of a radiolabel, a fluorescent label, a magnetic label, and an enzymatic label.

17. The kit of claim 16, wherein the label is selected from the group consisting of TAMRA (5[6]-carboxytetramethylrhodamine), ROX (5[6]-carboxy-X-rhodamine), JOE (6-carboxy-4′,5′-dichloro-2′,7′-dimethoxyfluorescein), FAM (5[6]-carboxyfluorescein), R110, R6G, TET, HEX, NAN, ZOE, VIC, NED, PET, BigDye, fluorescein, rhodamine, Cy2, Cy3, Cy5, Cy5.5 and Texas Red (sulphorhodamine 101 acid chloride).

18. A method of analyzing at least one polymorphic site in a biological sample comprising at least one single-stranded template, said method comprising the steps of:
a) combining the biological sample with a sequencing primer specific for each polymorphic site of interest in each template and a primer extension preparation, to form an assay mixture, wherein the preparation comprises:
i) chain elongating nucleotides lacking one nitrogenous base that is complementary to one polymorphic variant present in the template at the polymorphic site; and
ii) a template-dependent nucleic acid polymerase;
b) incubating the mixture for a time and at a temperature sufficient to extend the primer by addition of at least one nucleotide; and
c) determining the size of the primer after incubation.

19. The method of claim 18, wherein the template is immobilized on a solid phase.

20. The method of claim 18, wherein the template is in a solution.

21. The method of claim 18, wherein the chain elongating nucleotides comprise a single nitrogenous base.

22. The method of claim 18, wherein the primer or the chain elongating nucleotides are labeled.

23. The method of claim 22, wherein the label is selected from the group consisting of a radiolabel, a fluorescent label, a magnetic label, and an enzymatic label.

24. A kit for analyzing at least one polymorphic site in a biological sample comprising at least one single-stranded template, said kit comprising:
a) a sequencing primer specific for each polymorphic site of interest in the template;
b) a primer extension preparation comprising:
i) chain elongating nucleotides lacking one nitrogenous base that is complementary to one polymorphic variant present in the template at the polymorphic site;
ii) a template dependent nucleic acid polymerase.

25. The kit of claim 24, wherein the chain elongating nucleotides comprise a single nitrogenous base.

26. The kit of claim 24, further comprising a solid phase means for binding the template.

27. The kit of claim 24, wherein each primer further comprises a retention moiety selected from the group consisting of a polypeptide, an oligonucleotide, a polyamine, a polysaccharide, an aliphatic moiety comprising between one and fifteen carbon atoms, and an aromatic moiety.

28. The kit of claim 24, wherein the primer or the chain elongating nucleotides comprises a label.

29. The kit of claim 28, wherein the label is selected from the group consisting of a radiolabel, a fluorescent label, a magnetic label, and an enzymatic label.

30. The kit of claim 28, wherein the label is selected from the group consisting of TAMRA (5[6]-carboxytetramethylrhodamine), ROX (5[6]-carboxy-X-rhodamine), JOE (6-carboxy-4′,5′-dichloro-2′,7′-dimethoxyfluorescein), FAM (5[6]-carboxyfluorescein), R100, R6G, TET, HEX, NAN, ZOE, VIC, NED, PET, BigDye, fluorescein, rhodamine, Cy2, Cy3, Cy5, Cy5.5 and Texas Red (sulphorhodamine 101 acid chloride).
